(19) United States
(12) Patent Application Publication
(10) Pub. No.: US 2018/0291263 A1
(43) Pub. Date: Oct. 11, 2018

(54) SPIROFLUORENE DERIVATIVES AND ORGANIC ELECTROLUMINESCENT DEVICES

(71) Applicant: WUHAN CHINA STAR OPTOELECTRONICS TECHNOLOGY CO., LTD., Wuhan, Hubei (CN)

(72) Inventor: Biao PAN, Wuhan, Hubei (CN)

(21) Appl. No.: 15/567,378

(22) PCT Filed: May 18, 2017
(86) PCT No.: PCT/CN2017/084848

§ 371 Date: Oct. 18, 2017

(30) Foreign Application Priority Data
Mar. 1, 2017 (CN) 201710117468.2

(51) Int. Cl. C09K 11/06; C07D 209/86; C07D 235/18; C07F 9/53; H01L 51/00

(52) U.S. Cl. CPC C09K 11/06; C07D 209/86; C07D 235/18; H01L 51/5012; H01L 51/0056; H01L 51/0072; C07F 9/5325

(57) ABSTRACT

The present disclosure provides spirofluorene derivatives and organic electroluminescent devices comprising the spirofluorene derivatives as a host material. The spirofluorene derivative is represented by General Formula I. In the present disclosure, hole transporting performance and electron transporting performance of molecules can be adjusted through modifying 3,6 points of spirofluorene with groups that have hole transporting performance and electron transporting performance, so as to solve the problem that the traditional phosphorescent materials cannot simultaneously achieve high triplet energy level, carrier transfer matching, and high glass transition temperature.

### FIELD OF THE INVENTION

The present disclosure relates to the field of displays, and in particular, to spirofluorene derivatives and organic electroluminescent devices.

### BACKGROUND OF THE INVENTION

In 1987, Professors DENG Qingyun and Vanslyke made a bilayer organic electroluminescent device using Ultrathin Film Technology by using a transparent conducting film as an anode, AlQ3 as a luminescent layer, triarylamine as a hole transporting layer, and Mg/Ag alloy as a cathode (Appl. Phys. Lett., 1987, 52, 913). In 1990, Burroughes et al. invented an OLED having a luminescent layer made of conjugated polymers PPV (Nature. 1990, 347, 539). Since then, OLEDs have been a hot research topic all over the world.

Because of the influence of spin-forbidden reactions, most of what we usually see in daily life is a phenomenon of fluorescences. Researches on OLED technology were initially focused on fluorescent devices. However, according to the theory of spin quantum statistics, a maximum Internal Quantum Efficiency of a fluorescent electroluminescent device is only 25%, while that of a phosphorescent electroluminescent device can reach 100%. Accordingly, in 1999, Forrest and Thompson et al. (Appl. Phys. Let., 1999, 75, 4.) obtained a green OLED by doping a green phosphorescent material Ir(PPy)3 into a host material of 4,4′-N, N′-dicarbazole-biphenyl (CBP) at a concentration of 6 wt %. The maximum External Quantum Efficiency (EQE) of such a green OLED reaches 8%, which broke theoretical limit of electroluminescent devices. After that, phosphorescent luminescent materials have generated significant interest. Since then, phosphorescent electroluminescent materials and phosphorescent devices become a hot research topic all over the world.

There are three crucial factors for a good phosphorescent material: the first one is a sufficiently high triplet energy level (ET) to achieve effective energy transfer; the second one is a balanced carrier transport in devices for enhancing luminous efficiency of the devices; and the last one is a sufficiently high glass transition temperature (Tg) to ensure the stability of the devices at high current density for increasing the life of organic luminescent devices. In order to achieve these three different requirements simultaneously on one molecule, researchers carried out a number of meaningful attempts and developed different types of phosphorescent luminescent host materials.

Fluorene derivatives are a potential material among phosphorescent luminescent host materials due to their good thermodynamic stability and chemical stability as well as their extremely high fluorescence quantum efficiency. However, the fluorene derivatives usually have a lower triplet energy level, which is insufficient to meet the requirements of a blue phosphorescent host martial, so that its application on phosphorescent devices is restricted. For example, Lee et al. proposed a method of improving the triplet energy level and Tg of materials by changing fluorene to spirofluorene and modifying it at its 2, 7 points (Jang S E, Joo C W, Jeon S O, Yook K S, Lee J Y. The relationship between the substitution position of the diphenylphosphine oxide on the spirobifluorene and device performances of blue phosphorescent organic light-emitting diodes. Org Electron 2010; 11:1059-65.). This proves that spirofluorene may improve the triplet energy and Tg of materials. However, according to the calculation of quantum mechanics, modification at 2, 7 points is not the best solution.

As a result, it is necessary to provide a new spirofluorene derivative to solve the problems existing in the conventional technologies.

### SUMMARY OF THE INVENTION

The object of the present disclosure is to provide a new spirofluorene derivative and organic electroluminescent devices using such a new spirofluorene derivative. Hole transporting performance and electron transporting performance of molecules can be adjusted by modifying 3, 6 points of spirofluorene with groups that have hole transporting performance and electron transporting performance, so as to solve the problem that the traditional phosphorescent materials cannot simultaneously achieve high triplet energy level, carrier transfer matching and high glass transition temperature.

To achieve the above objects, the present disclosure first provides a spirofluorene derivative represented by the following General Formula I:

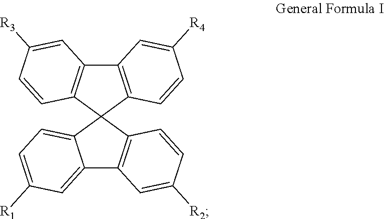

wherein R₁ and R₂ are both electron-transporting groups, and R₃ and R₄ are both hole-transporting groups.

In a preferred embodiment of the present disclosure, the electron-transporting group is selected from a group consisting of diphenylphosphoryl, m-phenyl-benzoimidazolyl group, and hydrogen, and the hole-transporting group is selected from a group consisting of carbazolyl and hydrogen.

In one embodiment of the present disclosure, the electron-transporting group is selected from a group consisting of hydrogen, cyano, diphenylphosphoryl, p-triphenylphosphynyl group, m-triphenylphosphynyl group, o-triphenylphosphynyl group, 2-pyridinyl, 3-pyridinyl, 4-pyridinyl, aza-9-carbazolyl, p-phenyl-benzoimidazolyl group, 4-N-benzimidazle, m-phenyl-benzoimidazolyl group, o-phenyl-benzoimidazolyl group, 3-N-benzimidazle, o-phenyl-1,3,4-oxadiazolyl group, m-phenyl-1,3,4-oxadiazolyl group, p-phenyl-1,3,4-oxadiazolyl group, o-phenyl-1,4,5-triazolyl group, m-phenyl-1,4,5-triazolyl group, p-phenyl-1,4,5-triazolyl group, o-triphenylphosphynyl group, 2-dioxodibenzothiophenyl, 3-dioxodibenzothiophenyl, 4-dioxodibenzothiophenyl, phenanthroimidazolyl, N-phenanthroimidazolyl, and p-phenyl-phenanthroimidazolyl group.

In one embodiment of the present disclosure, the hole-transporting group is selected from a group consisting of hydrogen, phenyl, p-methylphenyl group, 9-carbazolyl, tert-butyl-9-carbazolyl, aza-9-carbazolyl, diaza-9-carbazolyl, triphenylsilyl, triphenyiamine group, p-triphenylamine group, dimethyl-p-triphenylamine group, di-tert-butyl substituted carbazolyl group, 2-naphthyl substituted p-triphenylamine group, 3,6-di-tert-butyl-carbazolylphenyl, bis(3,6-di-tert-butyl-carbazolyl) substituted phenyl group, p-triphenylamine group, dimethyl-p-triphenylamine group, 1-naphthyl substituted p-triphenylamine group, 2-naphthyl substituted p-triphenylamine group, p-carbazolyl-phenyl group, (pyridyl-3-yl)carbazolyl, 2-dibenzothiophene, 3-dibenzothiophene, and 4-dibenzothiophene.

In one embodiment of the present disclosure, R₁ and R₂ represent the same or different substituent groups.

In one embodiment of the present disclosure, R₃ and R₄ represent the same or different substituent groups.

In a preferred embodiment of the present disclosure, each of R₁ and R₂ independently represents diphenylphosphoryl or m-phenyl-benzoimidazolyl group, and each of R₃ and R₄ independently represents hydrogen.

In a preferred embodiment of the present disclosure, each of R₁ and R₂ independently represents hydrogen, and each of R₃ and R₄ independently represents carbazolyl.

In a preferred embodiment of the present disclosure, there is provided a spirofluorene derivative represented by the following Formula i, ii, or iii:

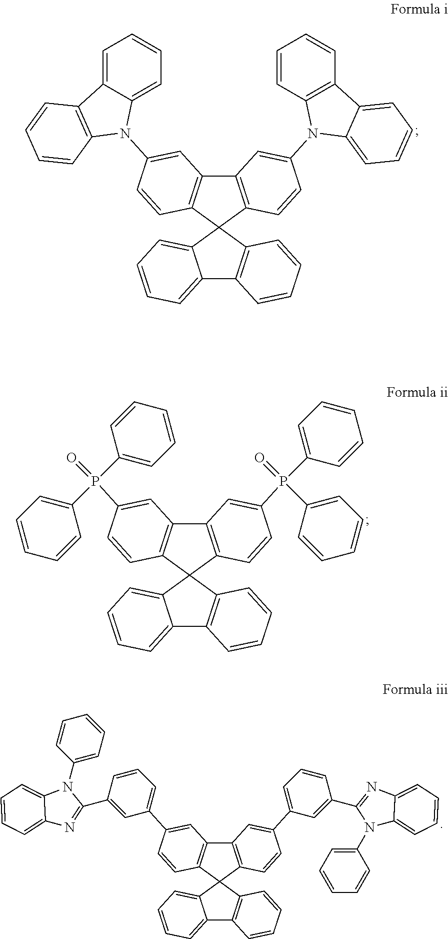 [SMILES: c1ccc2c(c1)-c1ccccc1C21c2ccc(-n3c4ccccc4c4ccccc43)cc2-c2cc(-n3c4ccccc4c4ccccc43)ccc21 ; O=P(c1ccccc1)(c1ccccc1)c1ccc2c(c1)-c1cc(P(=O)(c3ccccc3)c3ccccc3)ccc1C21c2ccccc2-c2ccccc21 ; c1ccc(-n2c(-c3cccc(-c4ccc5c(c4)-c4cc(-c6cccc(-c7nc8ccccc8n7-c7ccccc7)c6)ccc4C54c5ccccc5-c5ccccc54)c3)nc3ccccc32)cc1]

The present disclosure further provides an organic electroluminescent device using the above-mentioned spirofluorene derivative as a host material. In particular, the organic electroluminescent device provided in the present disclosure comprises at least one organic electroluminescent layer containing the spirofluorene derivative represented by the Formula i, ii, or iii.

In one embodiment of the present disclosure, the organic electroluminescent device comprises: a first electrode layer arranged on a substrate; one or more organic electroluminescent layers arranged on the first electrode layer, wherein the organic electroluminescent layer has a thickness of 15 to 25 nm and is made of the spirofluorene derivative doped with FIrpic; and, a second electrode layer arranged on the organic electroluminescent layer.

In a preferred embodiment of the present disclosure, doping ratio of the FIrpic is 5 to 10 wt %, preferably 7 wt %.

In one embodiment of the present disclosure, the organic electroluminescent device further comprises: an electron injecting layer sandwiched between the second electrode layer and the organic electroluminescent layer, an electron transporting layer sandwiched between the electron injecting layer and the organic electroluminescent layer, a hole injecting layer sandwiched between the first electrode layer and the organic electroluminescent layer, a hole transporting layer sandwiched between the hole injecting layer and the organic electroluminescent layer, and an exciton blocking layer sandwiched between the hole transporting layer and the organic electroluminescent layer.

In a preferred embodiment of the present disclosure, the electron injecting layer has a thickness of 0.5 to 1.5 nm, the electron transporting layer has a thickness of 30 to 50 nm, the hole injecting layer has a thickness of 5 to 15 nm, the hole transporting layer has a thickness of 50 to 70 nm, and the exciton blocking layer has a thickness of 2 to 10 nm.

In one embodiment of the present disclosure, the first electrode layer (anode) is formed of ITO, the hole injecting layer is formed of molybdenum trioxide, the hole transporting layer is formed of NPB, the exciton blocking layer is formed of mCP, the electron transporting layer is formed of TmPyPB, the electron injecting layer is formed of LiF, and the second electrode layer (cathode) is formed of Al.

The disclosure has the following advantages.

(1) The spirofluorene derivatives provided in the present disclosure have a higher triplet energy level to realize the energy transfer of the triplet excitons from the host to the guest.

(2) The spirofluorene derivatives provided in the present disclosure have a balanced carrier mobility to realize an effective recombination of holes and electrons in the light emitting region for increasing the luminous efficiency of the device.

(3) The spirofluorene derivatives provided in the present disclosure have a higher glass transition temperature and a better thermal stability, so that the service life of the light emitting device can be improved.

(4) OLED devices comprising a luminescent layer made of the spirofluorene derivative according to the present disclosure have an excellent performance, and the current efficiency, the power efficiency, and the external quantum efficiency thereof can achieve a high level in the performance of current blue phosphorescent devices.

(5) OLED devices comprising an electron transporting layer made of the spirofluorene derivative according to the present disclosure have a good stability within a large voltage range, which may effectively reduce the interfacial energy barrier between the electron transporting layer and the luminescent layer, avoid the interfacial charge accumulation and exciton quenching and help to increase the lifetime of devices, so that the OLED devices have a wide application prospect in the full color display field.

### BRIEF DESCRIPTION OF THE DRAWINGS

The above and other objects, features, and advantages of the present disclosure will be more apparent from the following detailed description taken in conjunction with the accompanying drawings, in which:

FIG. 1 is a fluorescence emission spectrum of the spirofluorene derivatives according to one embodiment of the present disclosure.

FIG. 2 is a low-temperature phosphorescence spectrum of the spirofluorene derivatives according to one embodiment of the present disclosure.

FIG. 3 is a diagram illustrating the glass transition temperature of the spirofluorene derivatives according to one embodiment of the present disclosure.

FIG. 4 is a UV absorption spectrum of the spirofluorene derivatives according to one embodiment of the present disclosure.

FIG. 5 is a structural schematic view of an organic electroluminescent device according to one embodiment of the present disclosure.

FIG. 6 is a diagram illustrating the energy level of an organic electroluminescent device according to one embodiment of the present disclosure.

FIG. 7 is a brightness-current density-voltage characteristic curve graph of the organic electroluminescent devices according to one embodiment of the present disclosure.

FIG. 8 is a current efficiency/power efficiency-brightness characteristic curve graph of the organic electroluminescent devices according to one embodiment of the present disclosure.

FIG. 9 is an electroluminescent spectrum of the organic electroluminescent devices according to one embodiment of the present disclosure.

### DESCRIPTION OF THE INVENTION

Hereinafter, some embodiments will be described in detail with reference to illustrative drawings. In the description of the elements of the present disclosure, terms such as “first”, “second”, “A”, “B”, “(a)”, “(b)”, and the like may be used. These terms are merely used to distinguish one component from other components, and the property, order, sequence, and the like of the corresponding component are not limited by the corresponding term. It should be noted that when it is described in the specification that one component is “connected”, “coupled”, or “joined” to another component, a third component may be “connected”, “coupled”, and “joined” between the first and second components, although the first component may be directly connected, coupled, or joined to the second component.

### Example 1. Spirofluorene Derivatives

A spirofluorene derivative is provided in the present embodiment, which is represented by the following General Formula I:

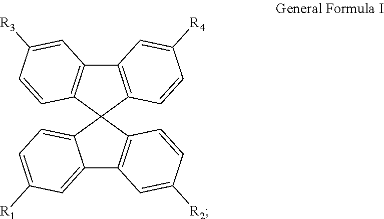

wherein R1 and R2 are both electron-transporting groups, and R3 and R4 are both hole-transporting groups.

The electron-transporting group includes, but is not limited to, hydrogen, cyano, diphenylphosphoryl, p-triphenylphosphynyl group, m-triphenylphosphynyl group, o-triphenylphosphynyl group, 2-pyridinyl, 3-pyridinyl, 4-pyridinyl, aza-9-carbazolyl, p-phenyl-benzoimidazolyl group, 4-N-benzimidazle, m-phenyl-benzoimidazolyl group, o-phenyl-benzoimidazolyl group, 3-N-benzimidazle, o-phenyl-1,3,4-oxadiazolyl group, m-phenyl-1,3,4-oxadiazolyl group, p-phenyl-1,3,4-oxadiazolyl group, o-phenyl-1,4,5-triazolyl group, m-phenyl-1,4,5-triazolyl group, p-phenyl-1,4,5-triazolyl group, o-triphenylphosphynyl group, 2-dioxodibenzothiophenyl, 3-dioxodibenzothiophenyl, 4-dioxodibenzothiophenyl, phenanthroimidazolyl, N-phenanthroimidazolyl, and p-phenyl-phenanthroimidazolyl group.

The specific structures and names of parts of the above-mentioned electron transport groups are listed below.

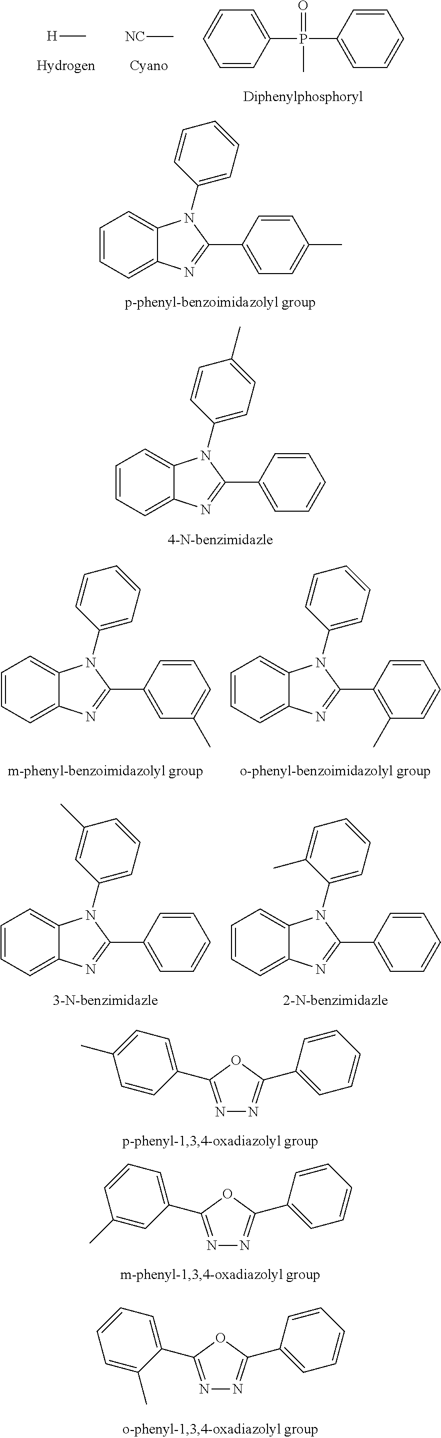

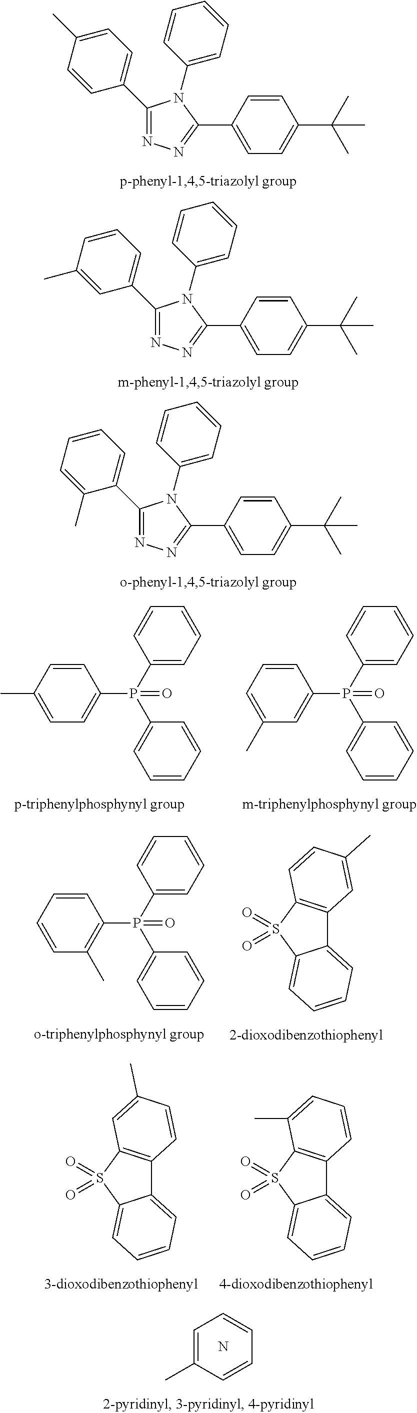

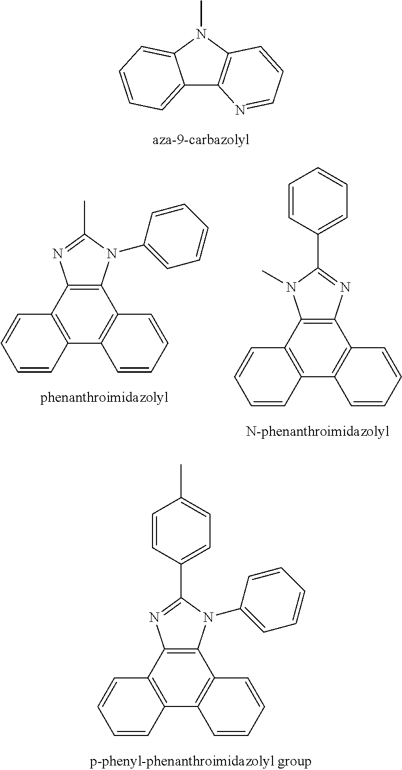 [SMILES: Cn1c2ccccc2c2ncccc21 ; Cc1nc2c3ccccc3c3ccccc3c2n1-c1ccccc1 ; Cn1c(-c2ccccc2)nc2c3ccccc3c3ccccc3c21 ; Cc1ccc(-c2nc3c4ccccc4c4ccccc4c3n2-c2ccccc2)cc1]

The hole-transporting group includes, but is not limited to, hydrogen, phenyl, p-methylphenyl group, 9-carbazolyl, tert-butyl-9-carbazolyl, aza-9-carbazolyl, diaza-9-carbazolyl, triphenylsilyl, triphenyiamine group, p-triphenylamine group, dimethyl-p-triphenylamine group, di-tert-butyl substituted carbazolyl group, 2-naphthyl substituted p-triphenylamine group, 3,6-di-tert-butyl-carbazolylphenyl, bis(3,6-di-tert-butyl-carbazolyl) substituted phenyl group, p-triphenylamine group, dimethyl-p-triphenylamine group, 1-naphthyl substituted p-triphenylamine group, 2-naphthyl substituted p-triphenylamine group, p-carbazolyl-phenyl group, (pyridyl-3-yl)carbazolyl, 2-dibenzothiophene, 3-dibenzothiophene, and 4-dibenzothiophene.

The specific structures and names of parts of the above-mentioned hole-transporting groups are listed below.

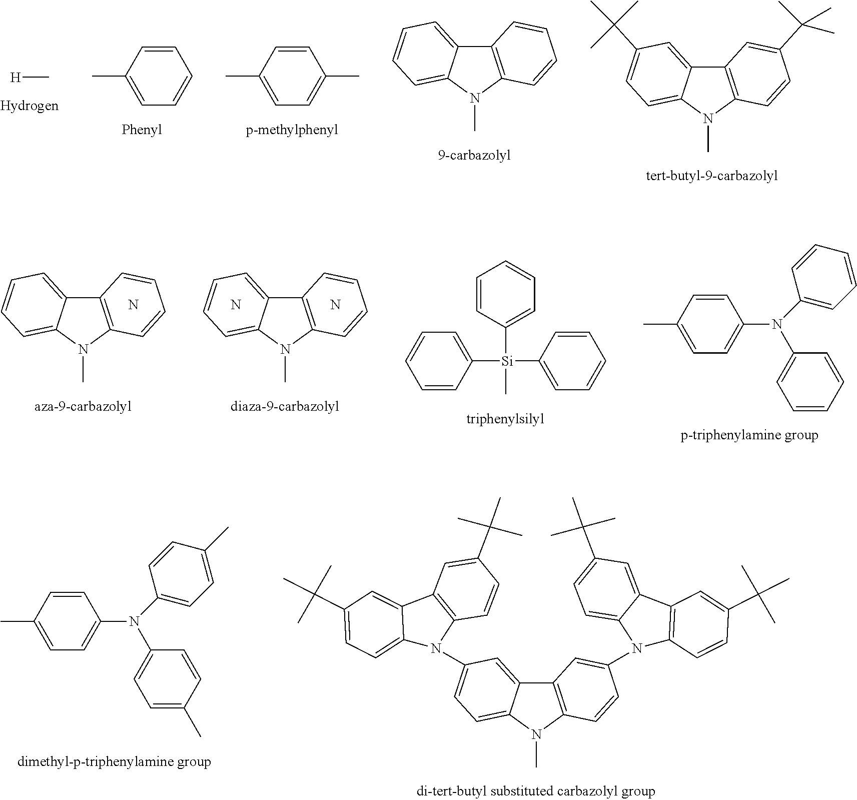

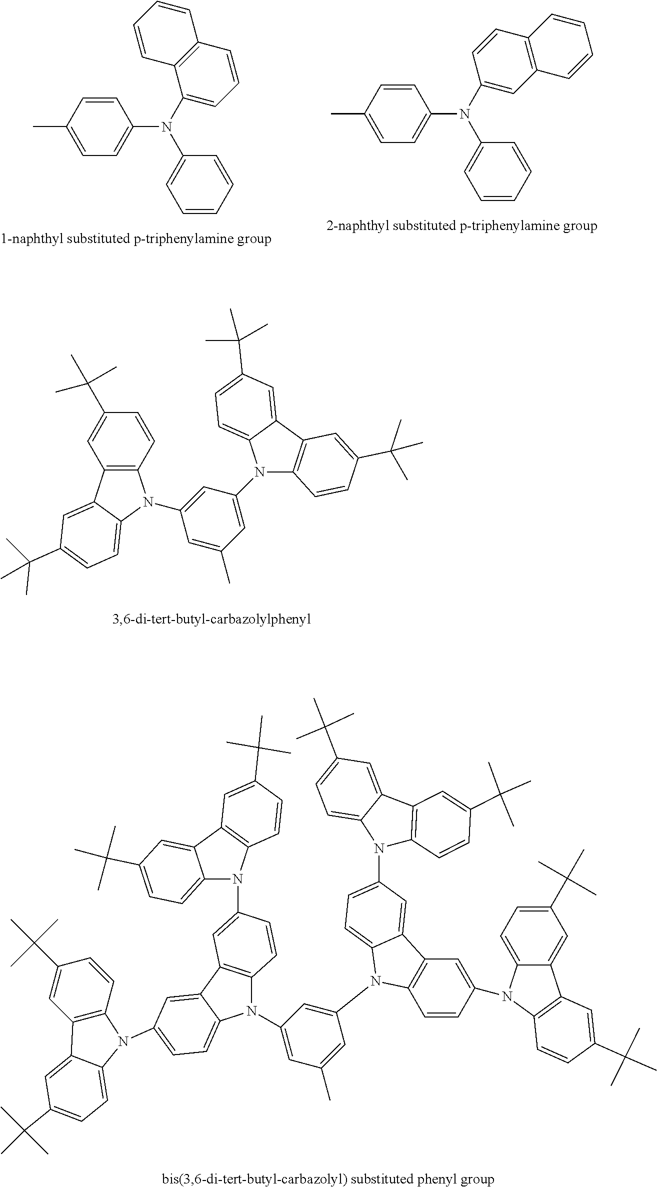

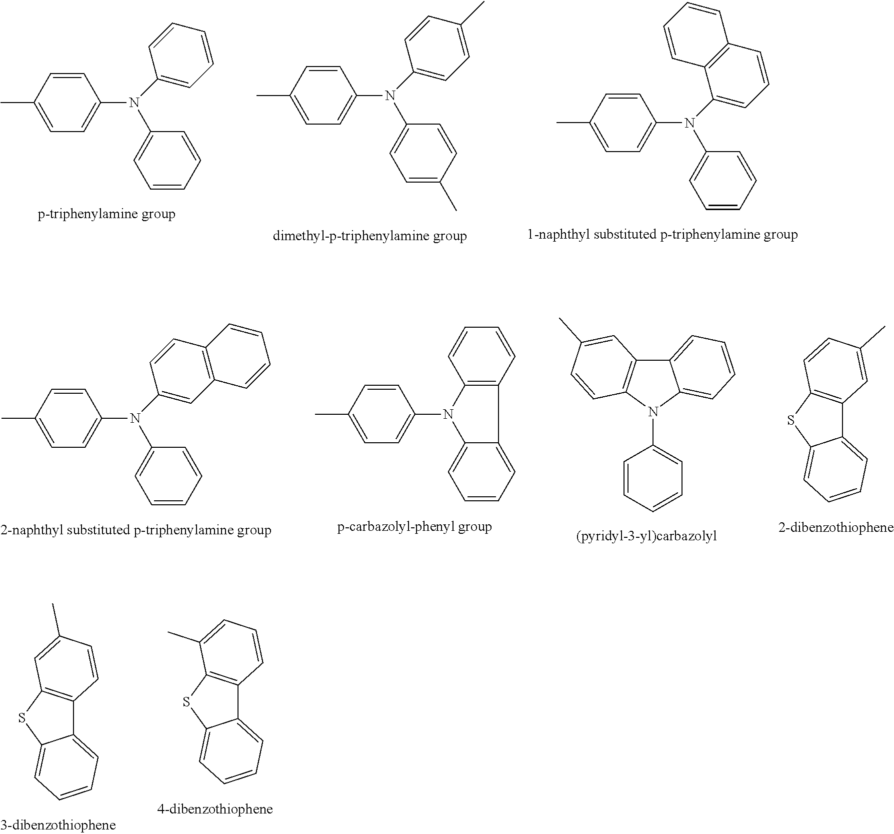

R1 and R2 may be the same or different substituent groups, and R3 and R4 may be the same or different substituent groups.

Optionally, each of R1 and R2 independently represents diphenylphosphoryl or m-phenyl-benzoimidazolyl group, while each of R3 and R4 independently represents hydrogen. Optionally, each of R1 and R2 independently represents hydrogen, while each of R3 and R4 independently represents carbazolyl.

### Example 2. Spirofluorene Derivative BSBDC

A spirofluorene derivative is provided in the present embodiment, which is represented by Formula i and is denoted as BSBDC:

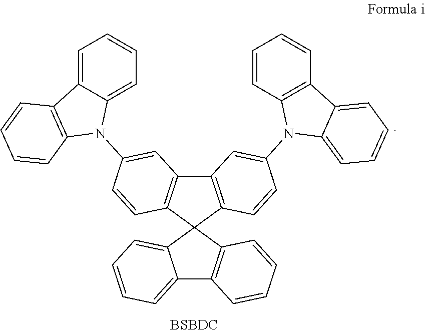

The preparation method is as follows.

### Step 1. Preparation of Intermediate 3,6-dibromoquinone

100 ml of nitrobenzene, 10.2 g (49.0 mmol) of phenanthrenequinone and 1.0 g (4.0 mmol) of dibenzoyl peroxide were added into a flask in turn, 5.0 ml (100 mmol) of liquid bromide was added while stirring followed by rapidly heating to 80° C. to react overnight. During the reaction, the reaction vessel was connected to an inverted triangular funnel and aqueous sodium hydroxide solution for tail gas absorption while preventing suction. After completion of the reaction, a yellow solid was filtered, washed with absolute ethanol and dried to obtain 14.8 g of crude product, which is the intermediate 3,6-dibromoquinolone. Yield: 85%. 1H-NMR: (CDCl3, 400 MHz): δ (ppm) 8.19 (m, 2H), 8.14˜8.12 (d, J=8.0 Hz, 2H), 7.74˜7.72 (d, J=8.0 Hz, 2H).

### Step 2. Preparation of Intermediate 3,6-dibromo-fluoren-9-one

60.0 g (1.0 mol) of potassium hydroxide, 400 ml of water, and 13.0 g (35.0 mmol) of 3,6-dibromoquinolone obtained in Step 1 were added into a 1000 ml flask in turn, followed by reacting at 100° C. for 3 hours. Then, 30.0 g (200.0 mmol) of potassium permanganate was added in portions and the reaction was continued for 10 hours. After completion of the reaction, the mixture was cooled down to room temperature. Subsequently, solid sodium thiosulfate powder was added to adjust the pH to neutral, and then black solids were precipitated out and filtered. The filter cake was wrapped in a filter paper and placed in a Soxhlet extractor to extract with dichloromethane for 3 days. Then, methylene chloride was evaporated to obtain 4.8 g of light yellow solid, which is the intermediate 3,6-dibromo-fluoren-9-one. Yield: 45%. 1H-NMR: (CDCl3, 400 MHz): δ (ppm) 7.74 (s, 2H), 7.60˜7.58 (d, J=8.0 Hz, 2H), 7.53˜7.51 (d, J=8.0 Hz, 2H).

### Step 3. Preparation of 3,6-dibromo-9,9′-spirobifluorene

First, 3.9 ml (50 mmol) of 2-bromobiphenyl was dissolved in 50 ml of tetrahydrofuran followed by being added dropwise to a 500 ml flask equipped with 2.8 g (117.0 mmol) of magnesium powder, and then several solid iodine particles were added to induce the reaction, to obtain a Grignard reagent.

Subsequently, the Grignard reagent was added to a solution of 3,6-dibromofluorenone (9.9 g, 55 mmol) in tetrahydrofuran (100 ml) through a double-end needle by using a pressure difference under the protection of nitrogen. After the transferring of the Grignard reagent was finished, the reaction was heated to reflux overnight and was stopped heating on the second day, and then naturally cooled to room temperature. 17.7 g of yellow crude product was obtained after recrystallization with N-hexane and filtration, yield is 76%.

Then, the crude product obtained above was dissolved in 50 ml of acetic acid, and 5 mol % of concentrated hydrochloric acid was added thereto, followed by heating to reflux at 120° C. overnight. After cooling to room temperature, the mixture was extracted with an organic solvent. The organic layer obtained by the extraction was dried over magnesium sulfate and spin-dried, and then was purified with silica gel column by using a mixed solution of ether/dichloromethane having a volume ratio of 3:1 to obtain 15.6 g of final product, which is 3,6-dibromo-9,9′-spirobifluorene. Yield is 73%. 1H-NMR: (CDCl3, 400 MHz): δ(ppm) 7.97 (s, 2H), 7.8 (d, J=8.0 Hz, 2H), 7.41 (t, J=1.2 Hz, 2H), 7.27 (t, J=4.0 Hz, 2H), 7.15 (t, J=1.6 Hz, 2H), 6.73 (d, J=8.0 Hz, 2H), 6.31 (d, J=8.0 Hz, 2H). MS (APCI): calcd for C25H14Br2:474.19, found, 475.4 (M+1)+.

### Step 4. Preparation of the Final Product BSBDC

800 mg (1.68 mmol) of 3,6-Dibromo-9,9′-spirobifluorene obtained in step 3, 842 mg (5.04 mmol) of carbazole, 342 mg (1.8 mmol) of cuprous iodide, 89 mg (3.4 mmol) of 18-Crown-6 and 1.25 g (9.07 mmol) of K2CO3 were sequentially dissolved in 2 ml of N,N-Dimethylpropyleneurea in a 50 ml round-bottomed flask, followed by heating under nitrogen to react at 180° C. for two days, and then the reaction solution was cooled to room temperature. The inorganic phase in the reaction solution was filtered off, followed by extraction with dichloromethane, and the organic layer was washed with water and then separated. The obtained organic layer was dried over anhydrous sodium sulfate, filtered and spin-dried to obtain the final product BSBDC, yield is 82%. 1H-NMR: (CDCl3, 400 MHz): δ (ppm) 8.14 (d, J=7.6 Hz, 2H), 8.01 (d, J=1.6 Hz, 4H), 7.93 (d, J=7.6 Hz, 2H), 7.52˜7.41 (m, 12H), 7.26 (m, 6H), 6.98 (dd, J=12.4 Hz, 4H). 13C-NMR (100 MHz, CDCl3): δ 150.47, 145.29, 143.73, 142.11, 140.67, 134.55, 130.76, 127.52, 126.22, 124.66, 122.17, 120.02, 119.89, 119.00, 109.83, 69.34. MS (APCI): calcd for C49H30N2, 646.78; found, 647.4 (M+1)+. Anal. calcd for C49H30N2(%): C, 90.99, H, 4.68, N, 4.33; found: C, 90.57, H, 4.38, N, 5.05.

### Example 3. Spirofluorene Derivative BSBDP

A spirofluorene derivative is provided in the present embodiment, which is represented by Formula ii and denoted as BSBDP:

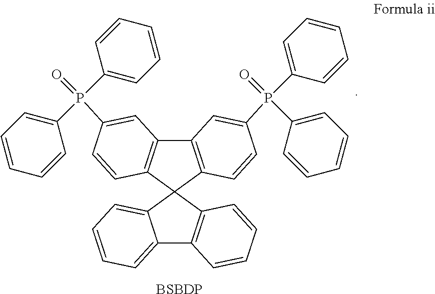

The preparation method is as follows.

### Step 1. Preparation of Intermediate 3,6-dibromoquinone

100 ml of nitrobenzene, 10.2 g (49.0 mmol) of phenanthrenequinone and 1.0 g (4.0 mmol) of dibenzoyl peroxide were added into a flask in turn, 5.0 ml (100 mmol) of liquid bromide was added while stirring followed by rapidly heating to 80° C. to react overnight. During the reaction, the reaction vessel was connected to an inverted triangular funnel and aqueous sodium hydroxide solution for tail gas absorption while preventing suction. After completion of the reaction, a yellow solid was filtered, washed with absolute ethanol and dried to obtain 14.8 g of crude product, which is the intermediate 3,6-dibromoquinolone. Yield: 85%. 1H-NMR: (CDCl3, 400 MHz): δ (ppm) 8.19 (m, 2H), 8.14˜8.12 (d, J=8.0 Hz, 2H), 7.74˜7.72 (d, J=8.0 Hz, 2H).

### Step 2. Preparation of Intermediate 3,6-dibromo-fluoren-9-one

60.0 g (1.0 mol) of potassium hydroxide, 400 ml of water and 13.0 g (35.0 mmol) of 3,6-dibromoquinolone obtained in Step 1 were added into a 1000 ml flask in turn, followed by reacting at 100° C. for 3 hours. Then, 30.0 g (200.0 mmol) of potassium permanganate was added in portions and the reaction was continued for 10 hours. After completion of the reaction, the mixture was cooled down to room temperature. Subsequently, solid sodium thiosulfate powder was added to adjust the pH to neutral, and then black solids were precipitated out and filtered. The filter cake was wrapped in a filter paper and placed in a Soxhlet extractor to extract with dichloromethane for 3 days. Then, methylene chloride was evaporated to obtain 4.8 g of light yellow solid, which is the intermediate 3,6-dibromo-fluoren-9-one. Yield: 45%. 1H-NMR: (CDCl3, 400 MHz): δ (ppm) 7.74 (s, 2H), 7.60˜7.58 (d, J=8.0 Hz, 2H), 7.53˜7.51 (d, J=8.0 Hz, 2H).

### Step 3. Preparation of 3,6-dibromo-9,9′-spirobifluorene

First, 3.9 ml (50 mmol) of 2-bromobiphenyl was dissolved in 50 ml of tetrahydrofuran followed by being added dropwise to a 500 ml flask equipped with 2.8 g (117.0 mmol) of magnesium powder, and then several solid iodine particles were added to induce the reaction, to obtain a Grignard reagent.

Subsequently, the Grignard reagent was added to a solution of 3,6-dibromofluorenone (9.9 g, 55 mmol) in tetrahydrofuran (100 ml) through a double-end needle by using a pressure difference under the protection of nitrogen. After the transferring of the Grignard reagent was finished, the reaction was heated to reflux overnight and was stopped heating till the second day, and then naturally cooled to room temperature. 17.7 g of yellow crude product was obtained after recrystallization with N-hexane and filtration, yield is 76%.

Then, the crude product obtained above was dissolved in 50 ml of acetic acid, and 5 mol % of concentrated hydrochloric acid was added thereto, followed by heating to reflux at 120° C. overnight. After cooling to room temperature, the mixture was extracted with an organic solvent. The organic layer obtained by the extraction was dried over magnesium sulfate and spin-dried, and then was purified with silica gel column by using a mixed solution of ether/dichloromethane having a volume ratio of 3:1 to obtain 15.6 g of final product, which is 3,6-dibromo-9,9′-spirobifluorene. Yield is 73%. 1H-NMR: (CDCl3, 400 MHz): δ (ppm) 7.97 (s, 2H), 7.8 (d, J=8.0 Hz, 2H), 7.41 (t, J=1.2 Hz, 2H), 7.27 (t, J=4.0 Hz, 2H), 7.15 (t, J=1.6 Hz, 2H), 6.73 (d, J=8.0 Hz, 2H), 6.31 (d, J=8.0 Hz, 2H). MS (APCI): calcd for C25H14Br2:474.19, found, 475.4 (M+1)+.

### Step 4. Preparation of the Final Product BSBDP

0.07 g (0.3 mmol) of Nickel(II) chloride hexahydrate, 0.4 g (2.0 mmol) of diphenylphosphine oxide, 0.39 g (6.0 mmol) of zinc powder, 0.09 g (0.6 mmol) of 2,2′-bipyridine, and 0.48 g (1.0 mmol) of 3,6-dibromo-9,9′-spirobifluorene obtained in step 3 were sequentially added into a 25 ml single-neck flask, and then 2 ml of N,N′-dimethylacetamide (DMAc) was added as a solvent. The reaction solution was heated under nitrogen to react at 120° C. for 48 hours. After completion of the reaction, the reaction solution was cooled to room temperature and then suction filtered to obtain an upper layer of solid, which was then washed with dichloromethane. Subsequently, the organic layer obtained by suction filtration was washed with water, dried over sodium sulfate and spin-dried, and then was purified with column to obtain the final product BSBDP, yield is 55%. 1H-NMR: (CDCl3, 400 MHz): δ (ppm) 8.03˜8.05 (d, J=7.6 Hz, 2H), 7.81 (d, J=7.6 Hz, 2H), 7.66 (m, 4H), 7.35˜7.56 (m, 18H), 7.04˜7.07 (t, J=7.6 Hz, 4H), 6.66 (t, J=1.6 Hz, 2H), 6.51 (m, 2H). 13C-NMR (100 MHz, CDCl3): δ 153.18, 147.08, 142.01, 141.34, 141.21, 133.02, 132.78, 132.53, 132.37, 132.27, 132.18, 132.01, 131.75, 129.02, 128.90, 128.78, 128.50, 128.23, 124.34 124.33, 124.26, 120.51, 65.64. MS (APCI): calcd for C49H34O2P2, 716.2; found, 717.5 (M+1)+.

### Example 4. Spirofluorene Derivative BSBDP

A spirofluorene derivative is provided in the present embodiment, which is represented by Formula iii and denoted as BSBDM:

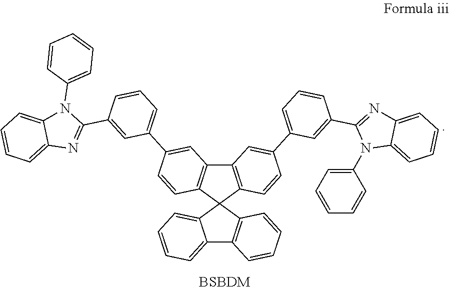

The preparation method is as follows.

### Step 1. Preparation of Intermediate 3,6-dibromoquinone

100 ml of nitrobenzene, 10.2 g (49.0 mmol) of phenanthrenequinone and 1.0 g (4.0 mmol) of dibenzoyl peroxide were added into a flask in turn, 5.0 ml (100 mmol) of liquid bromide was added while stirring followed by rapidly heating to 80° C. to react overnight. During the reaction, the reaction vessel was connected to an inverted triangular funnel and aqueous sodium hydroxide solution for tail gas absorption while preventing suction. After completion of the reaction, a yellow solid was filtered, washed with absolute ethanol and dried to obtain 14.8 g of crude product, which is the intermediate 3,6-dibromoquinolone. Yield: 85%. 1H-NMR: (CDCl3, 400 MHz): δ (ppm) 8.19 (m, 2H), 8.14˜8.12 (d, J=8.0 Hz, 2H), 7.74˜7.72 (d, J=8.0 Hz, 2H).

### Step 2. Preparation of Intermediate 3,6-dibromo-fluoren-9-one

60.0 g (1.0 mol) of potassium hydroxide, 400 ml of water and 13.0 g (35.0 mmol) of 3,6-dibromoquinolone obtained in Step 1 were added into a 1000 ml flask in turn, followed by reacting at 100° C. for 3 hours. Then, 30.0 g (200.0 mmol) of potassium permanganate was added in portions and the reaction was continued for 10 hours. After completion of the reaction, the mixture was cooled down to room temperature. Subsequently, solid sodium thiosulfate powder was added to adjust the pH to neutral, and then black solids were precipitated out and filtered. The filter cake was wrapped in a filter paper and placed in a Soxhlet extractor to extract with dichloromethane for 3 days. Then, methylene chloride was evaporated to obtain 4.8 g of light yellow solid, which is the intermediate 3,6-dibromo-fluoren-9-one. Yield: 45%. 1H-NMR: (CDCl3, 400 MHz): δ (ppm) 7.74 (s, 2H), 7.60˜7.58 (d, J=8.0 Hz, 2H), 7.53˜7.51 (d, J=8.0 Hz, 2H).

### Step 3. Preparation of 3,6-dibromo-9,9′-spirobifluorene

First, 3.9 ml (50 mmol) of 2-bromobiphenyl was dissolved in 50 ml of tetrahydrofuran followed by being added dropwise to a 500 ml flask equipped with 2.8 g (117.0 mmol) of magnesium powder, and then several solid iodine particles were added to induce the reaction, to obtain a Grignard reagent.

Subsequently, the Grignard reagent was added to a solution of 3,6-dibromofluorenone (9.9 g, 55 mmol) in tetrahydrofuran (100 ml) through a double-end needle by using a pressure difference under the protection of nitrogen. After the transferring of the Grignard reagent was finished, the reaction was heated to reflux overnight and was stopped heating till the second day, and then naturally cooled to room temperature. 17.7 g of yellow crude product was obtained after recrystallization with N-hexane and filtration, yield is 76%.

Then, the crude product obtained above was dissolved in 50 ml of acetic acid, and 5 mol % of concentrated hydrochloric acid was added thereto, followed by heating to reflux at 120° C. overnight. After cooling to room temperature, the mixture was extracted with an organic solvent. The organic layer obtained by the extraction was dried over magnesium sulfate and spin-dried, and then was purified with silica gel column by using a mixed solution of ether/dichloromethane having a volume ratio of 3:1 to obtain 15.6 g of final product, which is 3,6-dibromo-9,9′-spirobifluorene. Yield is 73%. 1H-NMR: (CDCl3, 400 MHz): δ (ppm) 7.97 (s, 2H), 7.8 (d, J=8.0 Hz, 2H), 7.41 (t, J=1.2 Hz, 2H), 7.27 (t, J=4.0 Hz, 2H), 7.15 (t, J=1.6 Hz, 2H), 6.73 (d, J=8.0 Hz, 2H), 6.31 (d, J=8.0 Hz, 2H). MS (APCI): calcd for C25H14Br2:474.19, found, 475.4 (M+1)+.

### Step 4. Preparation of the Final Product BSBDM

1.28 g (2.0 mmol) of 3,6-dibromo-9,9′-spirobifluorene obtained in step 3, 0.11 g (0.1 mmol) Pd(PPh3)4, 10.0 ml potassium carbonate with a molar concentration of 2.0 mol/L, 40 ml of toluene and 20 ml of ethanol were successively added to a 250 ml flask and sonicated for 15 minutes. The reaction solution was heated under nitrogen to react at 100° C. for 12 hours. 0.98 g of white solid powder were obtained after purification by column chromatography (ethyl acetate: petroleum ether=1:5), that is the final product BSBDM. Yield is 80%. 1H-NMR: (CDCl3, 400 MHz): δ (ppm) 7.93˜7.91 (d, J=8.0 Hz, 2H), 7.88˜7.85 (m, 6H), 5.69˜5.67 (m, 4H), 7.58˜7.53 (m, 6H), 7.49˜7.45 (m, 2H), 7.41˜7.34 (m, 8H), 7.29˜7.28 (m, 4H), 7.16˜7.12 (m, 2H), 7.09˜7.06 (m, 2H), 6.78˜6.73 (m, 4H). 13C-NMR: (CDCl3, 100 MHz): δ (ppm) 152.22, 148.50, 148.47, 143.01, 142.14, 141.77, 141.15, 140.20, 137.23, 137.20, 130.39, 130.07, 128.95, 128.68, 128.47, 128.44, 128.17, 127.94, 127.88, 127.61, 127.11, 124.28, 124.10, 123.48, 123.11, 120.09, 119.92, 118.76, 110.51, 65.49. MS (APCI): calcd for C63H40N₄, 852.3; found, 853.3. (M+1)+. Anal. calcd for C63H40N4: C, 88.71; H, 4.73; N, 6.57 found: C, 88.42; H, 4.63; N, 6.95.

### Example 5. Properties of Spirofluorene Derivatives BSBDC, BSBDP, and BSBDM

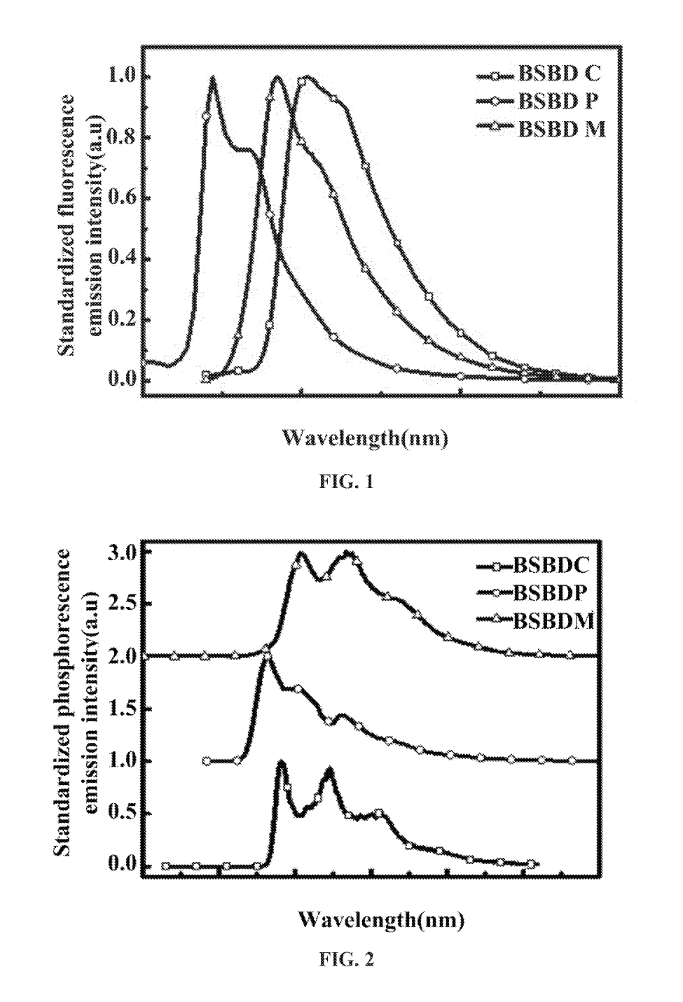

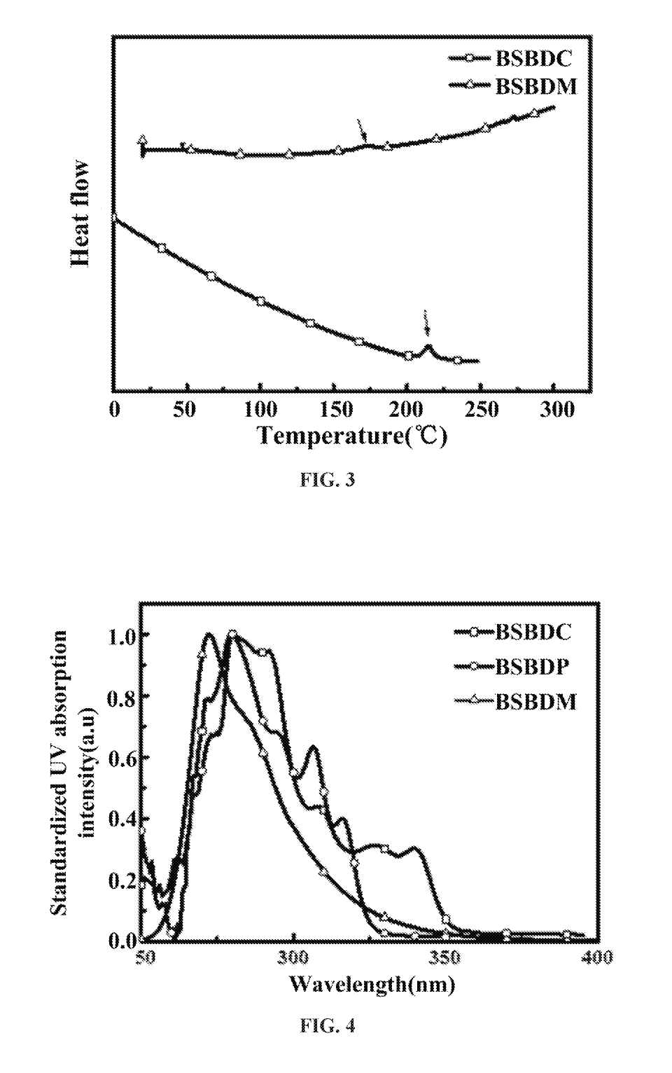

The Applicant studied the properties of the spirofluorene derivatives BSBDC, BSBDP, and BSBDM of the Examples 2, 3, and 4, and obtained a fluorescence emission spectrum as shown in FIG. 1, a low-temperature phosphorescence spectrum as shown in FIG. 2, glass transition temperatures as shown in FIG. 3, and a UV absorption spectrum as shown in FIG. 4.

FIG. 1 illustrates that each of the BSBDC, BSBDP, and BSBDM shows a certain absorption peak in the 250-400 nm band. The absorption peak of BSBDC at 285 nm is considered to be caused by π-π* transition of carbazole, and the simultaneous presence of two shoulder peaks can be attributed to π-π* transition of carbazole. For BSBDP, the maximum absorption wavelength is at 280 nm, which can be attributed to π-π* transition of the phosphorous oxygen double bonding in diphenylphosphoryl group. Likewise, the maximum absorption of BSBDM is at 272 nm, which can be attributed to π-π* charge transition in the benzimidazle group.

FIG. 2 illustrates that the sequence of the triplet energy level is BSBDP (2.87 eV)>BSBDC (2.81 eV)>BSBDM (2.73 eV).

FIG. 3 illustrates that the glass transition temperature of BSBDC reaches to 215° C. while that of BSBDM is 173° C.

The Eg of the three compounds can be calculated from FIG. 4, which is 3.48 eV (BSBDC), 3.78 eV (BSBDP), and 3.77 eV (BSBDM).

### Example 6. Organic Electroluminescent Device A

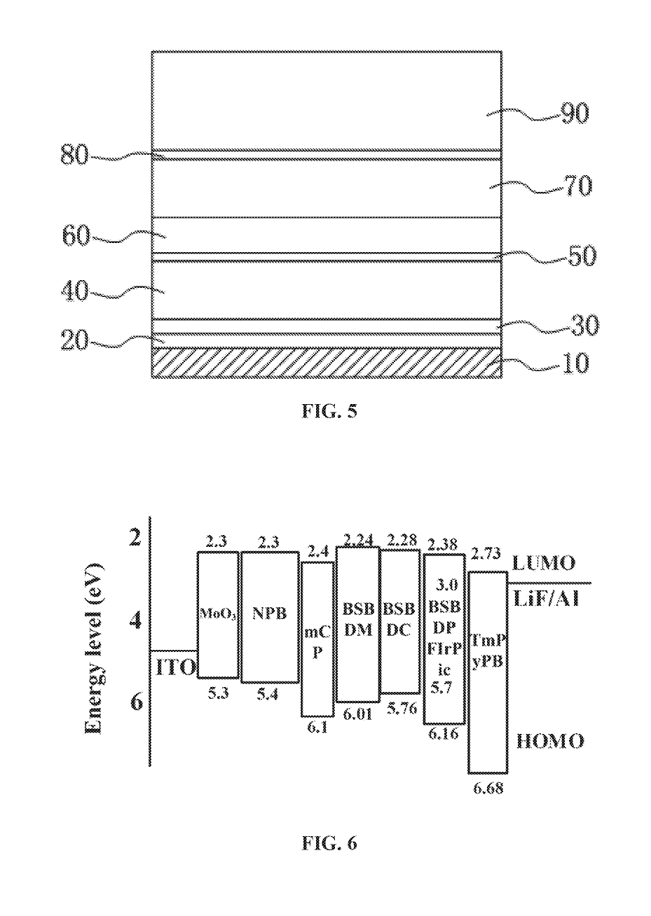

Referring now to FIG. 5, an organic electroluminescent device A is provided in the present embodiment, which comprises: a first electrode layer 20 arranged on a substrate 10, a hole injecting layer 30 arranged on the first electrode layer 20, a hole transporting layer 40 arranged on the hole injecting layer 30, an exciton blocking layer 50 arranged on the hole transporting layer 40, an organic electroluminescent layer 60 arranged on the exciton blocking layer 50 and made of the spirofluorene derivative BSBDC doped with FIrpic, an electron transporting layer 70 arranged on the organic electroluminescent layer 60, an electron injecting layer 80 arranged on the electron transporting layer 70, and a second electrode layer 90 arranged on the electron injecting layer 80.

The doping ratio of the FIrpic is 7 wt % in the present embodiment.

In the present embodiment, the first electrode layer 20 (anode) is formed of ITO, the hole injecting layer 30 is formed of molybdenum trioxide (MoO3), the hole transporting layer 40 is formed of NPB, the exciton blocking layer 50 is formed of mCP, the electron transporting layer 70 is formed of TmPyPB, the electron injecting layer 80 is formed of LiF, and the second electrode layer 90 (cathode) is formed of Al.

In the present embodiment, the hole injecting layer 30 has a thickness of 10 nm, the hole transporting layer 40 has a thickness of 60 nm, the exciton blocking layer 50 has a thickness of 5 nm, organic electroluminescent layer 60 has a thickness of 20 nm, the electron transporting layer 70 has a thickness of 40 nm, the electron injecting layer 80 has a thickness of 1 nm, and the second electrode layer 90 has a thickness of 100 nm.

Accordingly, the device structure of the organic electroluminescent device A in the present embodiment is as follows: ITO/MoO3 (10 nm)/NPB (60 nm)/mCP (5 nm)/BSBDC: 7 wt % FIrpic (20 nm)/TmPyPB (40 nm)/LiF (1 nm)/Al (100 nm). A diagram illustrating the energy level is shown in FIG. 6.

The organic electroluminescent device A is prepared by a known method. Such as, but not limited to, a method of cleaning a ITO glass for 30 minutes in both a cleaning agent and deionized water followed by drying in vacuum for 2 hours (105 V), and then putting the ITO glass into a plasma reactor for CFx plasma treatment for 1 minute followed by transferring it to a vacuum chamber to prepare an organic film and a metal electrode. The BSBDC is prepared as a host material by vacuum deposition for preparing the device.

### Example 7. Organic Electroluminescent Device B

An organic electroluminescent device B is provided in the present embodiment, which has a similar structure to the organic electroluminescent device A described in Example 6 and the difference is that the organic electroluminescent layer of the organic electroluminescent device B is made of the spirofluorene derivative BSBDP doped with FIrpic.

Accordingly, the device structure of the organic electroluminescent device B in the present embodiment is as follows: ITO/MoO3 (10 nm)/NPB (60 nm)/mCP (5 nm)/BSBDP: 7 wt % FIrpic (20 nm)/TmPyPB (40 nm)/LiF (1 nm)/Al (100 nm).

The organic electroluminescent device B is prepared by a known method. Such as, but not limited to, a method of cleaning a ITO glass for 30 minutes in both a cleaning agent and deionized water followed by drying in vacuum for 2 hours (105° C.)., and then putting the ITO glass into a plasma reactor for CFx plasma treatment for 1 minute followed by transferring it to a vacuum chamber to prepare an organic film and a metal electrode. The BSBDP is prepared as a host material by vacuum deposition for preparing the device.

### Example 8. Organic Electroluminescent Device C

An organic electroluminescent device C is provided in the present embodiment, which has a similar structure to the organic electroluminescent device A described in Example 6 and the difference is that the organic electroluminescent layer of the organic electroluminescent device C is made of the spirofluorene derivative BSBDM doped with FIrpic.

Accordingly, the device structure of the organic electroluminescent device C in the present embodiment is as follows: ITO/MoO3 (10 nm)/NPB (60 nm)/mCP (5 nm)/BSBDM: 7 wt % FIrpic (20 nm)/TmPyPB (40 nm)/LiF (1 nm)/Al (100 nm).

The organic electroluminescent device C is prepared by a known method. Such as, but not limited to, a method of cleaning a ITO glass for 30 minutes in both a cleaning agent and deionized water followed by drying in vacuum for 2 hours (105 V), and then putting the ITO glass into a plasma reactor for CFx plasma treatment for 1 minute followed by transferring it to a vacuum chamber to prepare an organic film and a metal electrode. The BSBDM is prepared as a host material by vacuum deposition for preparing the device.

### Example 9. Performance Verification of the Organic Electroluminescent Devices

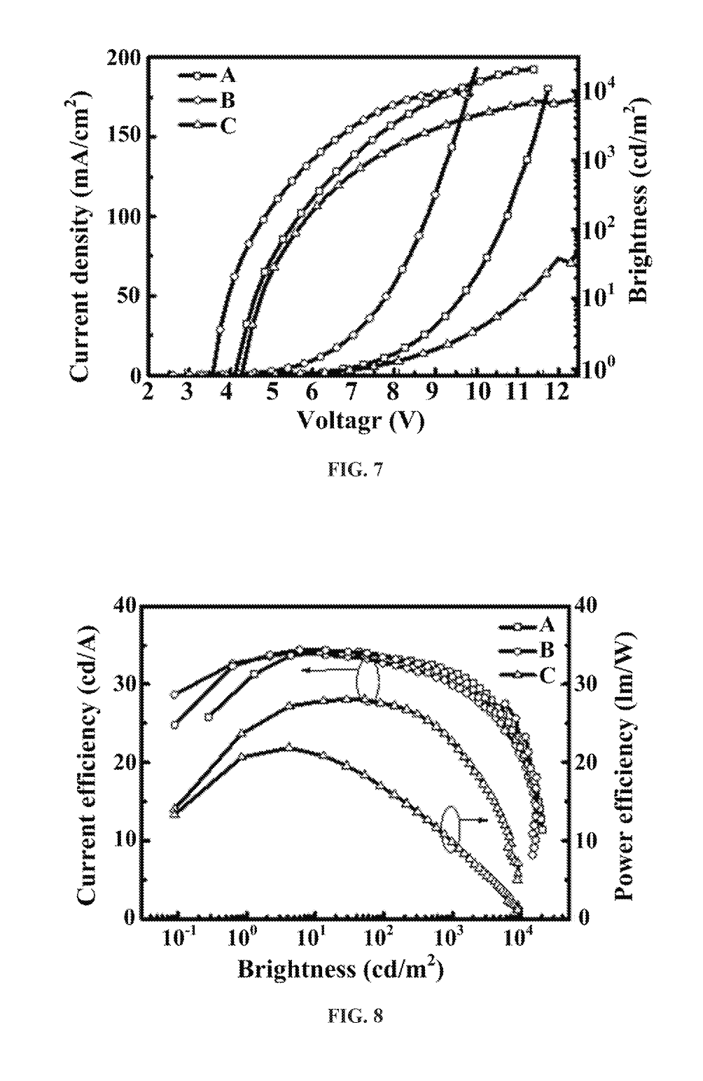

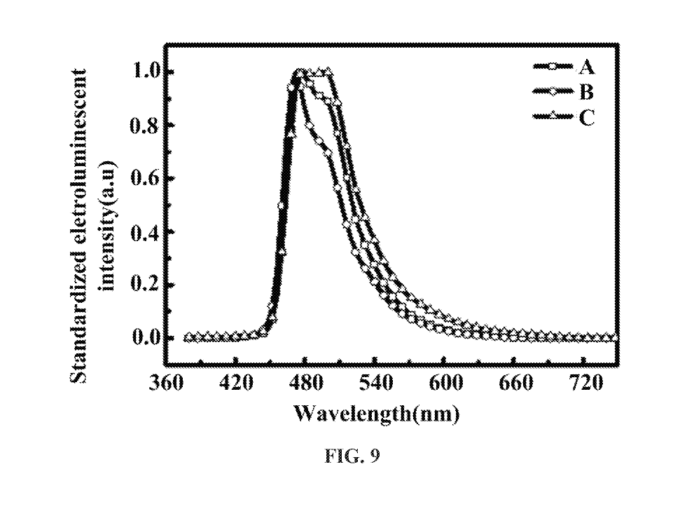

The Applicant further performed performance verification of the organic electroluminescent devices A to C obtained in Examples 6, 7, and 8, and obtained a brightness-current density-voltage characteristic curve graph as shown in FIG. 7, a current efficiency/power efficiency-brightness characteristic curve graph as shown in FIG. 8, and an electroluminescent spectrum as shown in FIG. 9.

FIG. 7 illustrates that the threshold voltage of the three devices is 3.2, 2.8, and 3.3 V, respectively. It can be seen that the threshold voltage of the three devices is about 3V, which proves the low energy barrier of carrier injection.

FIG. 8 illustrates that all three of these small molecular phosphorescent materials show good luminous efficiency under an evaporation condition, ηCE,max respectively reach 4.1, 34.2, and 28.1 cd/A, ηPE,max respectively reach 34.1, 34.4, and 22.3 lm/W, and maximum External Quantum Efficiency (EQE) respectively reach 16%, 18.7%, and 13.9%.

FIG. 9 illustrates that in the electroluminescence spectra of these three compounds, only two emission peaks were found at 476 nm and 500 nm, which were characteristic emission peaks of the guest material FIrpic. This shows that triplet excitons are completely transferred from the host to the guest, indicating that all three compounds BSBDC, BSBDP, and BSBDM described in the present disclosure can be used as a blue phosphorescent host material.

Thus, the invention has the following advantages.

(1) The spirofluorene derivatives provided in the present disclosure have a higher triplet energy level to realize the energy transfer of the triplet excitons from the host to the guest.

(2) The spirofluorene derivatives provided in the present disclosure have a balanced carrier mobility to realize an effective recombination of holes and electrons in the light emitting region for increasing the luminous efficiency of the device.

(3) The spirofluorene derivatives provided in the present disclosure have a higher glass transition temperature and a better thermal stability, so that the service life of the light emitting device can be improved.

(4) OLED devices comprising a luminescent layer made of the spirofluorene derivative according to the present disclosure have an excellent performance, and the current efficiency, the power efficiency, and the external quantum efficiency thereof can achieve a high level in the performance of current blue phosphorescent devices.

(5) OLED devices comprising an electron transporting layer made of the spirofluorene derivative according to the present disclosure have a good stability within a large voltage range, which may effectively reduce the interfacial energy barrier between the electron transporting layer and the luminescent layer, avoid the interfacial charge accumulation and exciton quenching, and help to increase the lifetime of devices, so that the OLED devices have a wide application prospect in the full color display field.

The present disclosure has been described with relative embodiments which are examples of the present disclosure only. It should be noted that the embodiments disclosed are not the limit of the scope of the present disclosure. Conversely, modifications to the scope and the spirit of the claims, as well as the equal of the claims, are within the scope of the present disclosure.

What is claimed is:

1. A spirofluorene derivative represented by the following General Formula I, 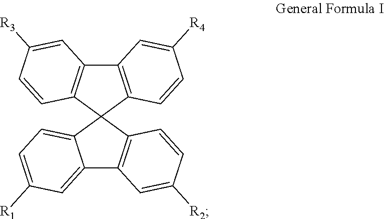
wherein R₁ and R₂ are both electron-transporting groups, and R₃ and R₄ are both hole-transporting groups, in which the electron-transporting group is selected from a group consisting of diphenylphosphoryl, m-phenyl-benzoimidazolyl group, and hydrogen, and the hole-transporting group is selected from a group consisting of carbazolyl and hydrogen.

2. The spirofluorene derivative according to claim 1, represented by the following Formula i, ii, or iii,

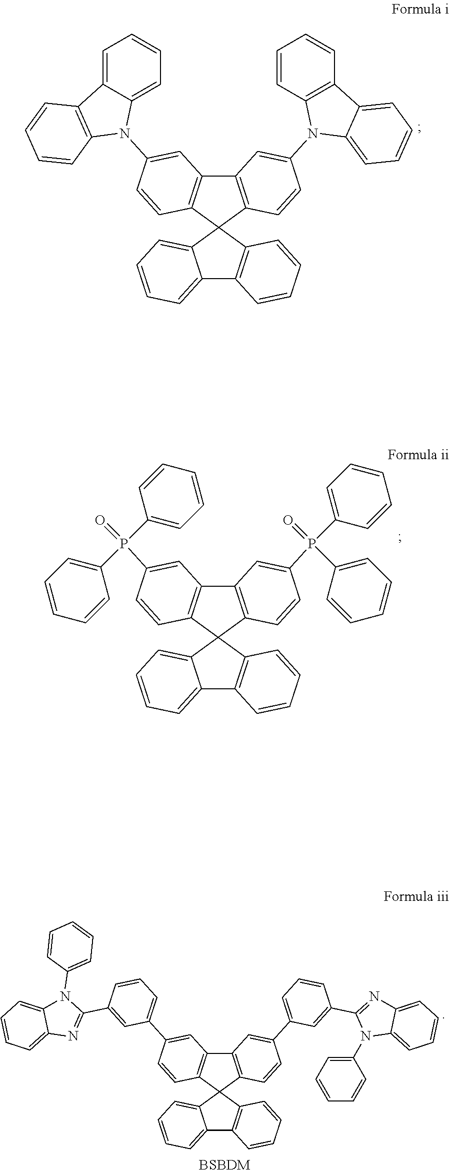

3. A spirofluorene derivative represented by the following General Formula I, 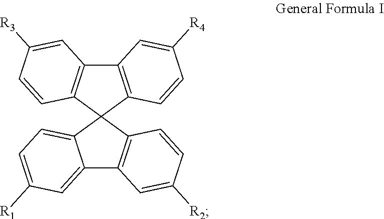
wherein R₁ and R₂ are both electron-transporting groups, and R₃ and R₄ are both hole-transporting groups.

4. The spirofluorene derivative according to claim 3, wherein the electron-transporting group is selected from a group consisting of hydrogen, cyano, diphenylphosphoryl, p-triphenylphosphynyl group, m-triphenylphosphynyl group, o-triphenylphosphynyl group, 2-pyridinyl, 3-pyridinyl, 4-pyridinyl, aza-9-carbazolyl, p-phenyl-benzoimidazolyl group, 4-N-benzimidazle, m-phenyl-benzoimidazolyl group, o-phenyl-benzoimidazolyl group, 3-N-benzimidazle, o-phenyl-1,3,4-oxadiazolyl group, m-phenyl-1,3,4-oxadiazolyl group, p-phenyl-1,3,4-oxadiazolyl group, o-phenyl-1,4,5-triazolyl group, m-phenyl-1,4,5-triazolyl group, p-phenyl-1,4,5-triazolyl group, o-triphenylphosphynyl group, 2-dioxodibenzothiophenyl, 3-dioxodibenzothiophenyl, 4-dioxodibenzothiophenyl, phenanthroimidazolyl, N-phenanthroimidazolyl, and p-phenyl-phenanthroimidazolyl group.

5. The spirofluorene derivative according to claim 4, wherein the hole-transporting group is selected from a group consisting of hydrogen, phenyl, p-methylphenyl group, 9-carbazolyl, tert-butyl-9-carbazolyl, aza-9-carbazolyl, diaza-9-carbazolyl, triphenylsilyl, triphenyiamine group, p-triphenylamine group, dimethyl-p-triphenylamine group, di-tert-butyl substituted carbazolyl group, 2-naphthyl substituted p-triphenylamine group, 3,6-di-tert-butyl-carbazolylphenyl, bis(3,6-di-tert-butyl-carbazolyl) substituted phenyl group, p-triphenylamine group, dimethyl-p-triphenylamine group, 1-naphthyl substituted p-triphenylamine group, 2-naphthyl substituted p-triphenylamine group, p-carbazolyl-phenyl group, (pyridyl-3-yl)carbazolyl, 2-dibenzothiophene, 3-dibenzothiophene and 4-dibenzothiophene.

6. The spirofluorene derivative according to claim 5, wherein R₁ and R₂ are the same or different substituent groups.

7. The spirofluorene derivative according to claim 5, wherein R₃ and R₄ are the same or different substituent groups.

8. The spirofluorene derivative according to claim 5, wherein each of R₁ and R₂ independently represents diphenylphosphoryl or m-phenyl-benzoimidazolyl group, and each of R₃ and R₄ independently represents hydrogen.

9. The spirofluorene derivative according to claim 5, wherein each of R₁ and R₂ independently represents hydrogen, and each of R₃ and R₄ independently represents carbazolyl.

10. An organic electroluminescent device comprising the spirofluorene derivative of claim 1 as a host material.

11. The organic electroluminescent device according to claim 10, wherein the organic electroluminescent device comprises:
a first electrode layer arranged on a substrate,
one or more organic electroluminescent layers arranged on the first electrode layer,
wherein the organic electroluminescent layer has a thickness of 15 to 25 nm and is made of the spirofluorene derivative doped with FIrpic, and
a second electrode layer arranged on the organic electroluminescent layer.

12. The organic electroluminescent device according to claim 11, wherein the organic electroluminescent device further comprises:
an electron injecting layer sandwiched between the second electrode layer and the organic electroluminescent layer,
an electron transporting layer sandwiched between the electron injecting layer and the organic electroluminescent layer,
a hole injecting layer sandwiched between the first electrode layer and the organic electroluminescent layer,
a hole transporting layer sandwiched between the hole injecting layer and the organic electroluminescent layer, and
an exciton blocking layer sandwiched between the hole transporting layer and the organic electroluminescent layer.

13. The organic electroluminescent device according to claim 12, wherein the electron injecting layer has a thickness of 0.5 to 1.5 nm, the electron transporting layer has a thickness of 30 to 50 nm, the hole injecting layer has a thickness of 5 to 15 nm, the hole transporting layer has a thickness of 50 to 70 nm, and the exciton blocking layer has a thickness of 2 to 10 nm.
